(19) United States
(12) Patent Application Publication
(10) Pub. No.: US 2018/0036420 A1
(43) Pub. Date: Feb. 8, 2018

(54) METHODS FOR ENHANCING THE DELIVERY OF MOLECULES ACROSS THE BLOOD BRAIN BARRIER

(71) Applicant: LAM Therapeutics, Inc., Guilford, CT (US)

(72) Inventors: Tian Xu, Guilford, CT (US); Henri Lichenstein, Guilford, CT (US); Jonathan M. Rothberg, Guilford, CT (US)

(21) Appl. No.: 15/666,700

(22) Filed: Aug. 2, 2017

Related U.S. Application Data
(60) Provisional application No. 62/370,442, filed on Aug. 3, 2016.

(51) Int. Cl. A61K 47/64; C12N 5/071; C07K 14/79

(52) U.S. Cl. CPC A61K 47/644; C07K 14/79; C12N 5/069; C07K 2317/55; C07K 2317/622

(57) ABSTRACT

The present invention relates to methods and compositions for enhancing the delivery of an agent across the blood brain barrier utilizing a composition comprising an inhibitor of PIKfyve.

### CROSS-REFERENCE TO RELATED APPLICATIONS

This application claims the benefit of priority from U.S. Provisional Application Ser. No. 62/370,442, filed on Aug. 3, 2016, the contents of which are hereby incorporated by reference in their entirety.

### FIELD OF THE INVENTION

The present invention relates to methods and compositions for enhancing the delivery of molecules, especially therapeutic agents, across the blood brain barrier.

### BACKGROUND OF THE INVENTION

The difficulty in getting molecules across the tight endothelial cell layer of the blood-brain barrier has presented a major challenge. The clinical use of biologics-based therapeutics, such as therapeutic antibodies and other protein-based therapies, as well as that of many small molecules, is often hampered or prevented by the inability to get sufficient amounts of these agents across the blood-brain barrier to achieve therapeutic levels in the target tissue of the brain.

Transcytosis is a receptor-mediated cellular process for moving molecules, including macromolecules, across the interior of a cell. The cells utilize receptor mediated endocytosis to bring the molecules into the cell, where they are then transported across the interior of the cell and exocytosed on the other side. This process is widely used, for example, by epithelial cells and some endothelial cells, such as capillaries of the blood brain barrier. It also occurs in other cells, such as neurons and the cells of the intestines.

The cellular process of transcytosis has been explored as a means for traversing the blood-brain barrier for the administration of therapeutic antibodies. Several studies in recent years have reported various levels of success utilizing antibodies against the transferrin receptor (TfR) or using a peptide as a transferrin ligand to shuttle the therapeutic antibody across the blood-brain barrier. However, it remains difficult to achieve therapeutic levels of antibody in the brain at least in part because high affinity TfR antibodies do not efficiently traverse the interior of the cell and instead are shunted into a degradation pathway. A recent paper by Niewoehner et al. demonstrates that bivalent binding of an antibody to the TfR induces lysosomal sorting and degradation consistent with the incomplete transcellular trafficking observed with bivalent therapeutic antibodies in vivo. See Neuron 81:49-60 (2014). The same group showed that a molecular shuttle using monovalent binding to the TfR increased brain penetration by an order of magnitude. Despite these advances, there remains a need to improve transcellular trafficking of therapeutic antibodies across the blood-brain endothelial barrier in order to achieve therapeutic levels of these antibodies in the brain. In addition, other molecules, including small molecule therapeutics, may also be moved by transcytosis. Accordingly, methods that facilitate transcytosis should benefit not only the delivery of therapeutic macromolecules to the brain, but small molecules as well.

PIKfyve is a phosphoinositide kinase that binds to PI(3)P and catalyzes the formation of the lipid second messengers PI(3,5)P₂ and PI(5)P. The lipid products PI(3,5)P₂ and PI(5)P in turn serve to establish membrane identity and control endolysosomal dynamics. PIKfyve is associated with the cytosolic leaflet of early endosomes and its activity is required for endomembrane homeostasis, endolysosomal function and proper retrograde transport from the endosome to the trans-Golgi network. Introduction of a kinase dead mutant into cells induces a swollen vacuole phenotype that can be rescued by the injection of PI(3,5)P₂. Inhibition of PIKfyve by pharmacological methods as well as RNAi also produces swollen vacuoles and disruption of endomembrane dynamics.

Apilimod, also referred to as STA-5326, hereinafter “apilimod”, is recognized as a potent transcriptional inhibitor of IL-12 and IL-23. See e.g., Wada et al. Blood 109 (2007): 1156-1164. In immune cells, the selective inhibition of IL-12/IL-23 transcription by apilimod was recently shown to be mediated by apilimod's direct binding to phosphatidylinositol-3-phosphate 5-kinase (PIKfyve). See, e.g., Cal et al. Chemistry and Biol. 20 (2013):912-921.

### SUMMARY OF THE INVENTION

The present invention is based, in part, upon Applicant's discovery that PIKfyve inhibitors can be utilized, either alone or in combination with other agents, to facilitate the delivery of agents, especially therapeutic agents, across the tight endothelial cell layer of the blood-brain barrier (referred to herein simply as ‘the blood-brain barrier’ or ‘BBB’). Accordingly, the invention provides methods and compositions for enhancing the delivery of agents, including small molecules as well as macromolecules, such as therapeutic antibodies and proteins, across the BBB. In certain embodiments, the invention also provides methods for facilitating transcytosis in a cell and methods for increasing the permeability of a cell layer. In accordance with these embodiments, the cell may be any cell, but is preferably an epithelial or endothelial cell, and most preferably a capillary endothelial cell. In one embodiment, the cell is a capillary endothelial cell of the BBB.

In one embodiment, the invention provides methods for enhancing the delivery of agents, including small molecules as well as macromolecules, such as therapeutic antibodies and proteins, across the BBB by contacting the cells of the BBB with an inhibitor of PIKfyve.

In one embodiment, the invention provides methods for enhancing the delivery of agents, including small molecules as well as macromolecules, such as therapeutic antibodies and proteins, across the BBB by combining anti-transferrin receptor antibody technology with an inhibitor of PIKfyve in order to increase the amount of the agent that is transcytosed across the barrier and/or decrease the amount of the agent that is lost to a degradation pathway.

In one embodiment, the invention provides a method for increasing the transcytosis of an agent across a cell, the method comprising contacting the cell with (i) a composition comprising the agent and (ii) an inhibitor of PIKfyve. In one embodiment, the composition comprising the agent is a pharmaceutical composition. In one embodiment, the composition comprising the agent also comprises a transferrin receptor binding moiety.

The invention also provides methods for administering a therapeutic agent to a subject in need thereof, the method comprising administering to the subject (i) a composition comprising the agent and a transferrin receptor binding moiety and (ii) an inhibitor of PIKfyve.

In accordance with the methods utilizing a composition comprising a transferrin receptor binding moiety, the transferrin receptor binding moiety may comprise or consist of an anti-transferrin receptor antibody, or an antigen-binding fragment thereof. In one embodiment, the transferrin receptor binding moiety comprises or consists of an anti-transferrin receptor monoclonal antibody or a single chain Fab fragment of an anti-transferrin receptor monoclonal antibody. In one embodiment, the monoclonal antibody is a chimeric antibody, e.g., a human chimeric anti-murine transferrin receptor monoclonal antibody.

In accordance with any of the methods described herein, the inhibitor of PIKfyve may be selected from any suitable inhibitor including, for example small molecule inhibitors such as apilimod, APY0201, and YM-201636, or RNA-based inhibitors such as small interfering RNAs (RNAi), or inhibitory antibodies, and antigen-binding fragments thereof. In one embodiment, the PIKfyve inhibitor is apilimod free base, or a pharmaceutically acceptable salt, solvate, clathrate, hydrate, polymorph, prodrug, analog or derivative thereof. In one embodiment, the PIKfyve inhibitor is apilimod free base or apilimod dimesylate. In one embodiment, the PIKfyve inhibitor is APY0201 or YM-201636.

In accordance with any of the methods described herein, the amount of the PIKfyve inhibitor used in the method is an amount effective to transiently inhibit cellular PIKfyve. In one embodiment, cellular PIKfyve is inhibited for a period of time ranging from 6 to 12 hours or from 12 to 72 hours.

Where the agent is a macromolecule, the macromolecule may be any suitable macromolecule, e.g., a protein, a nucleic acid, etc., as described infra. In one embodiment, the macromolecule is an antibody, a bioactive peptide, or a therapeutic bacteriophage. In one embodiment the macromolecule is an antibody, preferably a therapeutic antibody. In one embodiment, the antibody is a chimera of the macromolecule and a transferrin receptor antibody or an ApoB LDLR binding domain.

The methods described here can be used in to treat diseases and disorders treatable with the agent whose transport across the BBB is facilitated by the inhibition of PIKfyve as described herein.

In one embodiment, the invention provides a method for treating a subject in need of treatment for Alzheimer's disease. In accordance with this embodiment, the agent is a therapeutic agent. In one embodiment, the therapeutic agent is a therapeutic macromolecule selected from an anti-β-secretase (BACE1) antibody, an anti-amyloid-β antibody, an anti-tau protein antibody, or an antigen binding fragment of any of the foregoing. In one embodiment, the therapeutic antibody is administered intravenously at a dose of from 10 to 30 mg/kg, preferably about 20 mg/kg.

In one embodiment, the invention provides a method for treating a subject in need of treatment for brain cancer.

In accordance with any of the methods described herein, the inhibitor of PIKfyve may be administered prior to, at the same time as, or after the composition comprising the agent, and by the same or different route of administration. In some embodiments, the inhibitor of PIKfyve is formulated in a single dosage form with the composition comprising the agent. In other embodiments, the inhibitor of PIKfyve is formulated in a different dosage form.

The methods described herein also encompass administering to the subject at least one additional active agent, in addition to the inhibitor of PIKfyve and the composition comprising the agent. The at least one additional active agent may be administered in the same composition as the composition comprising inhibitor of PIKfyve, or in a separate composition. In one embodiment, the at least one additional active agent is a non-therapeutic agent. In one embodiment, the non-therapeutic agent is selected to ameliorate one or more side effects of the inhibitor of PIKfyve. In one embodiment, where the inhibitor of PIKfyve is an apilimod composition, preferably apilimod free base or apilimod dimesylate, the non-therapeutic agent is selected to ameliorate one or more side effects of the apilimod composition. In accordance with this embodiment, the non-therapeutic agent may be selected from the group consisting of ondansetron, granisetron, dolasetron and palonosetron; or from the group consisting of pindolol and risperidone. In one embodiment, the non-therapeutic agent is selected increase the bioavailability of the inhibitor of PIKfyve. In one embodiment, where the inhibitor of PIKfyve is an apilimod composition, preferably apilimod free base or apilimod dimesylate, the non-therapeutic agent is a CYP3A inhibitor, preferably ritonavir or cobicistat.

### BRIEF DESCRIPTION OF THE DRAWINGS

FIG. 1: Apilimod binds with high affinity to PIKfyve (Kd=75 pM).

FIG. 2: Immunostaining of the phosphoinositide PI(3,5)P₂ in H4 neuroglioma cells treated with the indicated concentration of apilimod for 24 hrs.

FIG. 3: Differential Interference Contrast (DIC) images of H4 neuroglioma cells treated with either vehicle (left) or 100 nM apilimod (right) for 24 hrs.)

FIG. 4: Left: DIC images of H4 transduced with either non-targeting shRNA (control) or hairpin targeted to the PIKFYVE gene (shPIKFYVE). Right: Viability of H4 cells after the induction of either control or PIKFYVE shRNA with doxycycline.

### DETAILED DESCRIPTION OF THE INVENTION

The present invention relates to compositions and methods for inhibiting cellular PIKfyve in order to facilitate the delivery of agents across the blood-brain barrier, for example by increasing the efficiency of transcytosis of an agent across a cell, preferably an endothelial cell of the blood-brain barrier, and/or increasing the permeability of the tight junction proteins of the barrier. In accordance with any of the methods described here, the agent to be delivered across the blood-brain barrier may be any agent, for example a small organic molecule or a macromolecule. The term “small organic molecule” refers to a low molecular weight (<900 daltons) organic compound. The term “macromolecule” refers to a molecule having in excess of 1,000 atoms, typically a nucleic acid or a protein (including antibodies), but may also refer to other complex molecules including carbohydrates and lipids. In one embodiment, the macromolecules are proteins. In one embodiment the proteins are antibodies. In the context of the present invention, the macromolecules are preferably therapeutic. In one aspect of the invention, the agent is a macromolecule, preferably a therapeutic macromolecule, such as a therapeutic antibody, or antigen-binding fragment thereof.

In one aspect, the present invention provides methods for increasing the permeability of a cell layer, preferably an epithelial or endothelial cell layer, and most preferably an endothelial cell layer of the BBB, by contacting the cells with an inhibitor of PIKfyve in an amount sufficient to increase the permeability.

In one aspect, the present invention provides methods for increasing the transcellular trafficking of an agent across a cell by contacting the cells with an inhibitor of PIKfyve in an amount sufficient to increase transcytosis of the agent. In one aspect, the invention provides methods of decreasing the amount of the agent that is shunted into a degradation pathway by contacting the cells with an inhibitor of PIKfyve in an amount sufficient to decrease the amount of the agent that is shunted into a degradation pathway. The method may further comprise the use of antibodies targeting the transferrin receptor (TfR), or antigen-binding fragments thereof, as molecular shuttles to move the agent across the blood brain barrier via transcytosis, in combination with the transient inhibition of cellular PIKfyve in the endothelial cells of the barrier.

The invention also provides methods for delivering an agent to the brain of a subject, preferably a human subject, and methods for increasing the amount of an agent delivered to the subject's brain, by administering a PIKfyve inhibitor to the subject, prior to, concurrently with, or after administration of the agent to the subject.

In accordance with one aspect of the present invention, inhibition of PIKfyve results in a decrease in lysosomal sorting and degradation in the endothelial cells of the blood-brain barrier, thereby reducing the amount of an agent, such as a therapeutic agent targeted to the brain, that is lost to degradation and increasing the amount that is successfully transported across the interior of the cell and exocytosed. The methods described herein can be used in combination with both high and low affinity transferrin receptor binding moieties as molecular shuttles for the agent, e.g., anti-TfR antibodies, and antigen binding fragments thereof, because inhibition of PIKfyve according to the methods described herein decreases the amount of these high affinity moieties that are lost to degradation pathways. In accordance with the present invention, the transcytosis of such moieties is enhanced by inhibiting PIKfyve, allowing for the achievement of higher levels of the macromolecules in the brain than in the absence of PIKfyve inhibition. In accordance with these methods, a cell encompasses any cell which utilizes transcytosis to move macromolecules. For example, the cell may be an epithelial cell, an endothelial cell, or a neuron. In one embodiment the cell is a capillary endothelial cell. In one embodiment, the cell is an endothelial cell of the BBB.

The invention also provides methods for increasing the amount of a therapeutic agent, including a therapeutic small molecule or macromolecule, such as a protein, e.g., a therapeutic antibody, in the brain of a subject following systemic delivery of the agent to the subject, for example by intravenous injection, the methods comprising administering a PIKfyve inhibitor to the subject prior to, concurrently with, or after systemic delivery of the agent to the subject. In one aspect, the PIKfyve inhibitor is administered in an amount effective to inhibit PIKfyve in the endothelial cells of the blood brain barrier of the subject.

In one embodiment, the invention provides a method for administering a therapeutic agent to the brain of a subject, preferably a human, in need thereof, the method comprising administering to the subject a composition comprising the therapeutic agent and a composition comprising a PIKfyve inhibitor. The composition comprising the PIKfyve inhibitor is administered in an amount effect to inhibit cellular PIKfyve activity in the cells of the subject, preferably in the endothelial cells of the blood-brain barrier. In one embodiment, PIKFyve activity is transiently inhibited. In one embodiment, PIKfyve activity is transiently inhibited in the endothelial cells of the blood-brain barrier in the subject. In one embodiment, the agent is a small molecule or a macromolecule. In one embodiment, the macromolecule is a therapeutic antibody.

In accordance with the methods of treatment described herein, the subject is preferably a human subject, but may be selected from other mammalian subjects including a rodent, such as a mouse, rat, or guinea pig, and a non-human primate.

In accordance with any of the embodiments described herein, the term “apilimod” refers to apilimod free base. The term “apilimod composition” is used to refer more generically to apilimod free base or a pharmaceutically acceptable salt, solvate, clathrate, hydrate, polymorph, prodrug, analog or derivative thereof. In accordance with any of the methods described herein, apilimod dimesylate may be used in place of apilimod free base.

In one aspect, the present invention provides a method comprising administering to the subject a therapeutically effective amount of an apilimod composition in combination with a macromolecule, for example a therapeutic antibody. In one embodiment, the apilimod composition comprises a pharmaceutically acceptable salt of apilimod. In one embodiment, the pharmaceutically acceptable salt of apilimod is apilimod dimesylate. In another embodiment, the apilimod composition comprises apilimod free base.

In accordance with any of the embodiments described herein, the macromolecule may be administered in a single dosage form with the apilimod or apilimod dimesylate, or with the apilimod composition, or in a separate dosage form.

In one embodiment, the method further comprises administering at least one additional active agent to the subject, in addition to the macromolecule and the apilimod, apilimod dimesylate, or apilimod composition. The at least one additional active agent may be a therapeutic agent or a non-therapeutic agent. The at least one additional active agent may be administered in a single dosage form with the apilimod, apilimod dimesylate, or apilimod composition, or in a separate dosage form.

In one embodiment, the at least one additional active agent is a non-therapeutic agent. In one embodiment, the non-therapeutic agent is selected to ameliorate one or more side effects of the apilimod, apilimod dimesylate, or apilimod composition. In one embodiment, the non-therapeutic agent is selected from the group consisting of ondansetron, granisetron, dolasetron and palonosetron. In one embodiment, the non-therapeutic agent is selected from the group consisting of pindolol and risperidone. In one embodiment, the non-therapeutic agent is selected to increase the bioavailability of the apilimod, apilimod dimesylate, or apilimod composition. In one aspect of this embodiment, the non-therapeutic agent is a CYP3A inhibitor. In a particular embodiment, the CYP3A inhibitor is ritonavir or cobicistat.

In one embodiment, the dosage form of the apilimod, apilimod dimesylate, or apilimod composition is an oral dosage form. In another embodiment, the dosage form is suitable for intravenous administration. In one embodiment, where the dosage form is suitable for intravenous administration, administration is by a single injection or by a drip bag.

As used herein, the term “an apilimod composition” may refer to a composition comprising apilimod itself (free base), or may encompass pharmaceutically acceptable salts, solvates, clathrates, hydrates, polymorphs, prodrugs, analogs or derivatives of apilimod, as described below. The structure of apilimod is shown in Formula I:

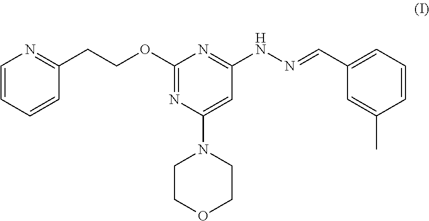 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

As used herein, the term “pharmaceutically acceptable salt,” is a salt formed from, for example, an acid and a basic group of an apilimod composition. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In one embodiment, the salt of apilimod comprises methanesulfonate.

The term “pharmaceutically acceptable salt” also refers to a salt prepared from an apilimod composition having an acidic functional group, such as a carboxylic acid functional group, and a pharmaceutically acceptable inorganic or organic base.

The term “pharmaceutically acceptable salt” also refers to a salt prepared from an apilimod composition having a basic functional group, such as an amino functional group, and a pharmaceutically acceptable inorganic or organic acid.

The salts of the compounds described herein can be synthesized from the parent compound by conventional chemical methods such as methods described in Pharmaceutical Salts: Properties, Selection, and Use, P. Hemrich Stahl (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

As used herein, the term “polymorph” means solid crystalline forms of a compound of the present invention (e.g., 2-[2-Pyridin-2-yl)-ethoxy]-4-N′-(3-methyl-benzilidene)-hydrazino]-6-(morpholin-4-yl)-pyrimidine) or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention (e.g., 2-[2-Pyridin-2-yl)-ethoxy]-4-N′-(3-methyl-benzilidene)-hydrazino]-6-(morpholin-4-yl)-pyrimidine) or a salt thereof, which further includes a stoichiometric or non-stoichiometric amount of water bound by non-covalent intermolecular forces.

As used herein, the term “clathrate” means a compound of the present invention (e.g., 2-[2-Pyridin-2-yl)-ethoxy]-4-N′-(3-methyl-benzilidene)-hydrazino]-6-(morpholin-4-yl)-pyrimidine) or a salt thereof in the form of a crystal lattice that contains spaces (e.g., channels) that have a guest molecule (e.g., a solvent or water) trapped within.

As used herein, the term “prodrug” means a derivative of a compound described herein (e.g., 2-[2-Pyridin-2-yl)-ethoxy]-4-N′-(3-methyl-benzilidene)-hydrazino]-6-(morpholin-4-yl)-pyrimidine) that can hydrolyze, oxidize, or otherwise react under biological conditions (in vitro or in vivo) to provide a compound of the invention. Prodrugs may only become active upon such reaction under biological conditions, or they may have activity in their unreacted forms. Examples of prodrugs contemplated in this invention include, but are not limited to, analogs or derivatives of a compound described herein (e.g., 2-[2-Pyridin-2-yl)-ethoxy]-4-N′-(3-methyl-benzilidene)-hydrazino]-6-(morpholin-4-yl)-pyrimidine) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

As used herein, the term “solvate” or “pharmaceutically acceptable solvate,” is a solvate formed from the association of one or more solvent molecules to one of the compounds disclosed herein (e.g., 2-[2-Pyridin-2-yl)-ethoxy]-4-N′-(3-methyl-benzilidene)-hydrazino]-6-(morpholin-4-yl)-pyrimidine). The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

### Antibodies

As described above, the compositions of the invention may comprise or consist of antibodies, for example therapeutic antibodies, anti-transferrin receptor antibodies, and anti-PIKfyve antibodies.

In one aspect, the antibodies for use in the methods of the invention are monoclonal antibodies. The terms “antibody” and “antibodies” refer to fully human antibodies, humanized antibodies, camelised antibodies, chimeric antibodies, CDR-grafted antibodies, single-chain Fvs (scFv), disulfide-linked Fvs (sdFv), Fab fragments, F(ab′) fragments, and antigen-binding fragments of any of the foregoing. In particular, the antibodies include immunoglobulin molecules and antigen-binding active fragments of immunoglobulin molecules, i.e., molecules that contain an antigen binding site. Such fragments may or may not be fused to another immunoglobulin domain including, but not limited to, an Fc region or fragment thereof. The skilled person will appreciate that other fusion products may be generated, including but not limited to, scFv-Fc fusions, variable region (e.g., VL and VH)-Fc fusions, and scFv-scFv-Fc fusions. Immunoglobulin molecules can be of any type, including, IgG, IgE, IgM, IgD, IgA and IgY, and of any class, including IgG₁, IgG₂, IgG₃, IgG₄, IgA₁ and IgA₂), or of any subclass. Preferably, the monoclonal antibodies for use in the methods and compositions of the invention are IgG antibodies.

Where the antibody is a monoclonal antibody, the monoclonal antibody may be a chimeric, human, or humanized antibody, or an antigen-binding fragments thereof. In one embodiment, the antibody is a monoclonal human or humanized antibody, or an antigen-binding fragement thereof. A monoclonal antibody is derived from a substantially homogeneous population of antibodies specific to a particular antigen, which population contains substantially similar epitope binding sites. Such antibodies may be of any immunoglobulin class including IgG, IgM, IgE, IgA, and any subclass thereof. Methods for monoclonal antibody production are well known in the art. Preferably, a monoclonal antibody for use in the methods and compositions of the invention is produced using hybridoma technology.

Antigen-binding fragments of the antibodies include, for example, Fab, Fab′, F(ab′)2 and Fv fragments. These fragments lack the heavy chain constant fragment (Fc) of an intact antibody and are sometimes preferred because they tend to clear more rapidly from the circulation and have less non-specific binding than an intact antibody. Such fragments are produced from intact antibodies using methods well known in the art, for example by proteolytic cleavage with enzymes such as papain (to produce Fab fragments) or pepsin (to produce F(ab′)₂ fragments). Preferably, an antigen-binding fragment is a dimer of heavy chains (a camelised antibody), a single-chain Fvs (scFv), a disulfide-linked Fvs (sdFv), a Fab fragment, or a F(ab′) fragment.

A human antibody is one in which all of the sequences arise from human genes. Human antibodies include antibodies having the amino acid sequence of a human immunoglobulin and include antibodies isolated from human immunoglobulin libraries or from mice that express antibodies from human genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring, which express human antibodies. The transgenic mice are immunized in the normal fashion with a selected antigen. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.) and Genpharm (San Jose, Calif.) can be engaged to provide human antibodies directed against a selected antigen using technology similar to that described above.

Human antibodies can also be derived from phage display of human antibody fragments. In phage display methods, functional antibody domains are displayed on the surface of phage particles, which carry the polynucleotide sequences encoding them. In particular, DNA sequences encoding variable heavy and variable light domains are amplified from animal cDNA libraries (e.g., human or murine cDNA libraries of lymphoid tissues). The DNA encoding the variable heavy and variable light domains are recombined together with an scFv linker by PCR and cloned into a phagemid vector. The vector is electroporated in E. coli and the E. coli is infected with helper phage. The phage used in these methods are typically filamentous phage including fd and M13. Phage expressing an antigen binding domain that binds to the antigen epitope of interest can be selected or identified with antigen, e.g., using labeled antigen or antigen bound or captured to a solid surface or bead. Examples of phage display methods include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Adv. Immunol. 57:191-280; International Application No. PCT/GB91/01134; International Application Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108. Preferably, after phage selection, the antibody coding regions from the phage are isolated and used to generate whole antibodies, including human antibodies as described in the above references.

A humanized antibody is an antibody which comprises a framework region having substantially the same amino acid sequence as human receptor immunoglobulin and a complementarity determining region (“CDR”) having substantially the same amino acid sequence as a non-human donor immunoglobulin. A humanized antibody comprises substantially all of at least one, and typically two, variable domains (Fab, Fab′, F(ab′)₂, Fv) in which all or substantially all of the CDR regions correspond to those of the non-human donor immunoglobulin (i.e., the donor antibody) and all or substantially all of the framework regions of the human acceptor immunoglobulin. The acceptor may comprise or consist of a consensus sequence of human immunoglobulins. Preferably, a humanized antibody also comprises at least a portion of an immunoglobulin constant region (Fc), typically that of a human immunoglobulin. Ordinarily, the antibody will contain a light chain and at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. The framework and CDR regions of a humanized antibody need not correspond precisely to the donor and acceptor sequences, e.g., the donor CDR or the acceptor framework may be mutagenized by substitution, insertion or deletion of at least one residue. Such mutations, however, will not be extensive. Usually, at least 75% of the humanized antibody residues will correspond to those of the acceptor framework and donor CDR sequences, more often 90%, and most preferably greater than 95%. A humanized antibody can be produced using variety of techniques known in the art, including but not limited to, CDR-grafting (see e.g., European Patent No. EP 239,400; International Publication No. WO 91/09967; and U.S. Pat. Nos. 5,225,539, 5,530,101, and 5,585,089), veneering or resurfacing (see e.g., European Patent Nos. EP 592,106 and EP 519,596; Padlan, 1991, Mol. Immunol. 28:489-498; Studnicka et al., 1994, Prot. Eng. 7:805-814; and Roguska et al., 1994, Proc. Natl. Acad. Sci. U.S.A. 91:969-973), chain shuffling (see e.g., U.S. Pat. No. 5,565,332), and techniques disclosed in, e.g., U.S. Pat. Nos. 6,407,213, 5,766,886, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55:5973s-5977s, Couto et al., 1995, Cancer Res. 55:1717-22, Sandhu, 1994, Gene 150:409-10, and Pedersen et al., 1994, J. Mol. Biol. 235:959-73. Often, framework residues in the framework regions will be substituted with the corresponding residue from the donor antibody to alter, preferably improve, antigen binding. These framework substitutions are identified by methods well known in the art, e.g by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties).

A chimeric antibody comprises non-human variable region sequences and human constant region sequences. A chimeric antibody may be monovalent, divalent or polyvalent. A monovalent chimeric antibody is a dimer formed by a chimeric heavy chain associated through disulfide bridges with a chimeric light chain. A divalent chimeric antibody is a tetramer formed by two heavy-light chain dimers associated through at least one disulfide bridge. A polyvalent chimeric antibody can also be produced, for example, by employing a heavy chain constant region that aggregates (e.g., from an IgM heavy chain).

A “camelised” antibody is one having a functional antigen binding site comprising only the heavy chain variable domains (VH), rather than the conventional antigen binding site which comprises both the heavy and the light chain variable domains (VL). Preferably, a camelised antibody comprises one or two VH domains and no VL domains. Preferably, a camelised antibody comprises two VH domains. Methods for making camelised antibodies are known in the art. See, for example, Riechmann et al., J. Immunol. Methods, 1999 231:25-38, and U.S. Patent Application Publication Nos. US 2004137570 and US 2004142432.

### Transferrin Receptor Binding Moiety

In some embodiments, the methods described herein comprise contacting a cell or administering to a subject in need thereof, a composition comprising a macromolecule and a transferrin receptor binding moiety, along with an inhibitor of PIKfyve. Transferrin receptor binding moieties are known in the art and include, for example, anti-transferrin receptor antibodies and fragments thereof.

In one embodiment, the transferrin receptor binding moiety binds to the transferrin receptor with high affinity. In another embodiment, the transferrin receptor binding moiety binds to the transferrin receptor with low affinity. Affinity is a measure of the strength of binding between two moieties, e.g., an antibody and an antigen. Affinity can be expressed in several ways. One way is in terms of the dissociation constant (K_{d}) of the interaction. K_{d} can be measured by routine methods, include equilibrium dialysis or by directly measuring the rates of antigen-antibody dissociation and association, the k_{off} and kₒₙ rates, respectively (see e.g., Nature, 1993 361:186-87). The ratio of k_{off}/kₒₙ cancels all parameters not related to affinity, and is equal to the dissociation constant K_{d} (see, generally, Davies et al., Annual Rev Biochem, 1990 59:439-473). Thus, a smaller K_{d} means a higher affinity. Another expression of affinity is Kₐ, which is the inverse of K_{d}, or kₒₙ/k_{off}. Thus, a higher Kₐ means a higher affinity. A high affinity antibody for use in the compositions and methods of the invention is an antibody that binds to an antigen of B. anthracis with a K_{d} in the picomolar (pM, 10⁻¹² M) or nanomolar (nM, 10⁻⁹ M) range.

In one embodiment, the transferrin receptor binding moiety binds to the transferrin receptor with a K_{d} of from 1 to 100 pM, from 100 to 250 pM, from 250 to 500 pM, or from 500 to 1000 pM. In another embodiment, the transferrin receptor binding moiety binds to the transferrin receptor with a K_{d} from 1 to 100 nM, from 100 to 250 nM, from 250 to 500 nM, or from 500 to 1000 nM, preferably, with a K_{d} from 1 to 200 pM or from 1 to 200 nM.

In another embodiment, the transferrin receptor binding moiety binds to the transferrin receptor with an affinity constant (Kₐ) of at least 10⁷M⁻¹, preferably with a Kₐ of from 10⁷M⁻¹ to 10⁸M⁻¹, from 10⁸M⁻¹ to 10⁹M⁻¹, from 10⁹M⁻¹ to 10¹⁰M⁻¹, or from 10¹⁰ M⁻¹ to 10¹¹M⁻¹.

### Methods of Treatment

The invention provides methods of treating a subject in need thereof with a therapeutic agent by enhancing the delivery of the agent to the brain via inhibition of PIKfyve in the endothelial cells of the blood-brain barrier. The methods comprise administering to the subject a composition comprising the agent along with an inhibitor of PIKfyve.

The term “enhancing delivery” in this context refers to increasing the accumulation of the macromolecule in the brain, e.g., in the brain parenchyma, and/or decreasing the amount of the macromolecule shunted into a degradation pathway, rather than a transcytosis pathway. Preferably, the amount of macromolecule delivered to the brain using the methods described here is sufficient to reach a therapeutic level of the macromolecule in the brain.

In one embodiment, the methods comprise administering to the subject a composition comprising both the agent and a transferrin receptor binding moiety, as described in detail above, along with an inhibitor of PIKfyve. The agent may be covalently or non-covalently bound to the transferrin receptor binding moiety. Thus, in one aspect, the methods comprising administering the agent along with a means to effect transferrin receptor mediated transcytosis (via the transferrin receptor binding moiety) and also inhibiting cellular PIKfyve, particularly inhibiting cellular PIKfyve in the endothelial cells of the blood brain barrier.

In accordance with the methods described herein, a “subject in need of” is a subject having a disease, disorder or condition, or a subject having an increased risk of developing a disease, disorder or condition relative to the population at large. The subject in need thereof can be one that is “non-responsive” or “refractory” to a currently available therapy for the disease or disorder. In this context, the terms “non-responsive” and “refractory” refer to the subject's response to therapy as not clinically adequate to relieve one or more symptoms associated with the disease or disorder. While the term “subject” includes any mammal, e.g., a human, primate, vertebrate, bird, mouse, rat, fowl, dog, cat, cow, horse, goat, camel, sheep or a pig, preferably the mammal is a human. The term “patient” as used herein refers to a human subject.

In one embodiment, the subject in need of treatment via the methods described herein is a human subject having Alzheimer's disease. In another embodiment, the subject is a human or animal subject having brain cancer.

In the context of the methods described herein, the amount of the composition comprising the agent administered to the subject is preferably a therapeutically effective amount. The term “therapeutically effective amount” refers to an amount sufficient to treat, ameliorate a symptom of, reduce the severity of, or reduce the duration of the disease or disorder being treated, or enhance or improve the therapeutic effect of another therapy, or sufficient to exhibit a detectable therapeutic effect in the subject.

In the context of the methods described herein, the amount of the PIKfyve inhibitor administered to the subject is the amount effective to inhibit PIKfyve kinase activity in the cells of the subject. Preferably, the cells are the endothelial cells of the blood brain barrier. In one embodiment, PIKfyve is inhibited transiently, for a period of time ranging from 12 to 48 hours.

An effective amount of a PIKfyve inhibitor can range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See, e.g., U.S. Pat. No. 7,863,270, incorporated herein by reference.

In more specific aspects, where the PIKfyve inhibitor is an apilimod composition, the inhibitor is administered at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). In one embodiment, an apilimod composition is administered at a dosage regimen of 100-1000 mg/day for 2, 4, 8. or 16 weeks. Alternatively or subsequently, an apilimod composition is administered at a dosage regimen of 100 mg-300 mg twice a day for 2, 4, 8, or 16 weeks, or optionally, for 52 weeks. Alternatively or subsequently, an apilimod composition is administered at a dosage regimen of 50 mg-1000 mg twice a day for 2, 4, 8, or 16 weeks, or optionally, for 52 weeks.

An effective amount of the PIKfyve inhibitor can be administered once daily or twice daily, or from two to five times daily, up to two times or up to three times daily, or up to eight times daily. In one embodiment, the PIKfyve inhibitor is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In one embodiment, the PIKfyve inhibitor is administered once or twice daily in a regimen of 5 days on, 2 days off in a 28-day cycle for a period of time that is from 1-2 months, from 1-3 months, from 1-4 months, from 1-5 months, from 1-6 months, or from 6-12 months.

The present invention also provides a monotherapy for the treatment of a disease, disorder or condition as described herein. As used herein, “monotherapy” refers to the administration of a single active or therapeutic compound to a subject in need thereof.

As used herein, “treatment”, “treating” or “treat” describes the management and care of a patient for the purpose of combating a disease, condition, or disorder and includes the administration of a therapeutic composition or compositions to alleviate the symptoms or complications of a disease, condition or disorder, or to eliminate the disease, condition or disorder.

As used herein, “prevention”, “preventing” or “prevent” describes reducing or eliminating the onset of the symptoms or complications of the disease, condition or disorder and includes the administration of a therapeutic composition or compositions to reduce the onset, development or recurrence of symptoms of the disease, condition or disorder.

In one embodiment, the administration of a therapeutic composition or compositions leads to the elimination of a symptom or complication of the disease or disorder being treated, however, elimination is not required. In one embodiment, the severity of the symptom is decreased. In the context of Alzheimer's disease, such symptoms may include clinical markers of severity or progression.

### Combination Therapy

The methods described herein also encompass combination therapy. As used herein, “combination therapy” or “co-therapy” includes the administration of a therapeutically effective amount of a PIKfyve inhibitor with a composition comprising a therapeutic agent. The methods may also comprising the administration of at least one additional active agent, as part of a specific treatment regimen intended to provide a beneficial effect from the co-action of the PIKfyve inhibitor with the therapeutic agent and the additional active agent. “Combination therapy” is not intended to encompass the administration of two or more therapeutic compounds as part of separate monotherapy regimens that incidentally and arbitrarily result in a beneficial effect that was not intended or predicted.

The at least one additional active agent may be a therapeutic agent or a non-therapeutic agent, and combinations thereof. With respect to therapeutic agents, the beneficial effect of the combination includes, but is not limited to, pharmacokinetic or pharmacodynamic co-action resulting from the combination of therapeutically active compounds. With respect to non-therapeutic agents, the beneficial effect of the combination may relate to the mitigation of a toxicity, side effect, or adverse event associated with a therapeutically active agent in the combination, or the beneficial effect may relate to increasing the bioavailability and/or therapeutic index of the therapeutic agent.

In one embodiment, the at least one additional agent is a non-therapeutic agent which mitigates one or more side effects of an apilimod composition, the one or more side effects selected from any of nausea, vomiting, headache, dizziness, lightheadedness, drowsiness and stress. In one aspect of this embodiment, the non-therapeutic agent is an antagonist of a serotonin receptor, also known as 5-hydroxytryptamine receptors or 5-HT receptors. In one aspect, the non-therapeutic agent is an antagonist of a 5-HT₃ or 5-HT₁ₐ receptor. In one aspect, the non-therapeutic agent is selected from the group consisting of ondansetron, granisetron, dolasetron and palonosetron. In another aspect, the non-therapeutic agent is selected from the group consisting of pindolol and risperidone.

In the context of combination therapy, administration of the PIKfyve inhibitor and the composition comprising the therapeutic agent may be simultaneous with or sequential to the administration of the one or more additional active agents. In another embodiment, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present invention.

The one or more additional active agents can be formulated for co-administration with the PIKfyve inhibitor or the composition comprising the therapeutic agent in a single dosage form; or the one or more additional active agents can be administered separately. When the additional active agent is administered separately, it can be by the same or a different route of administration as the PIKfyve inhibitor and/or the composition comprising the therapeutic agent.

Preferably, the administration of the PIKfyve inhibitor and/or the composition comprising the therapeutic agent in combination with the one or more additional agents provides a synergistic response in the subject being treated. In this context, the term “synergistic” refers to the efficacy of the combination being more effective than the additive effects of either single therapy alone. The synergistic effect of a combination therapy according to the invention can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

“Combination therapy” also embraces the administration of the PIKfyve inhibitor and the composition comprising the therapeutic agent in further combination with non-drug therapies. Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

### Pharmaceutical Compositions and Formulations

The present invention provides compositions that are preferably pharmaceutically acceptable compositions suitable for use in a mammal, preferably a human. In this context, the compositions may further comprise at least one pharmaceutically acceptable excipient or carrier.

In one embodiment, the composition comprises a PIKfyve inhibitor selected from apilimod free base, apilimod dimesylate, APY0201, and YM-201636.

In one embodiment, the PIKfyve inhibitor is combined with at least one additional active agent in a single dosage form. In one embodiment, the composition further comprises an antioxidant.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

In one embodiment, the unit dosage form comprises 25, 50, 75, 100, 200, or 300 milligrams of the active pharmaceutical ingredient, e.g., apilimod free base or apilimod dimesylate. In one embodiment, the unit dosage form is the form of a tablet or capsule for oral delivery.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

The following non-limiting examples are provided to further illustrate embodiments of the invention disclosed herein. It will be appreciated by those of skill in the art that the techniques disclosed in the examples that follow represent approaches that have been found to function well in the practice of the invention and thus can be considered to constitute examples of modes for its practice. However, those of skill in the art will, in light of the present disclosure, appreciate that many changes can be made in the specific embodiments that are disclosed and still obtain a like or similar result without departing from the spirit and scope of the invention.

### Example 1: Apilimod is a Highly Selective Binder of PIKfyve Kinase

The cellular target of apilimod was identified in whole cell lysate prepared from human neuroglioma cells. apilimod binding partners were identified using chemical capture mass spectrometry (CCMS, Caprotec Bioanalytics GmbH, Berlin Germany. See Michaelis et al., J. Med. Chem., 55 3934-44 (2012) and references cited therein). Briefly, two capture compound variants employing apilimod as selectivity function attached in a single orientation were synthesized and analyzed by LC-MS and 1H-NMR to ensure identity and purity. Capture conditions were optimized in whole cell lysate, e.g. minimization of non-specific interactions of the proteins with capture compounds, concentration of reagents and proteins to obtain maximum binding of proteins and capture compounds, etc. One capture compound was selected to identify specific protein binders in the CCMS experiments using apilimod as a competitor ligand. Proteins that are detected by LC-S in the capture assay and that are significantly diminished in competition control experiments are considered to be specific binders. These specific binders were further subjected to stringent data analysis criteria to determine specificity after unbiased data evaluation. Specific protein binders were ranked according to their fold change (FC) values in the capture experiments.

Only two proteins were identified as high probability candidate target proteins of apilimod: PIKfyve and Vac14. FC and p-values for these proteins in the four different capture compound concentration experiments are shown in Table 1.

TABLE 1

| | | Capture Compound | | | |
|---|---|---|---|---|---|
| | | Concentrations | | | |
| | | 0.1 μM | 0.5 μM | 1.0 μM | 2.0 μM |
| PIKfyve | log₂ (FC) | 6.3 | 6.2 | 4.1 | 4.3 |
| | −log₁₀ (p-value) | 3.7 | 2.8 | 5.1 | 3.9 |
| Vac14 | log₂ (FC) | 6.2 | 5.6 | Inf. | 3.9 |
| | −log₁₀ (p-value) | 3.9 | 3.8 | 1.9 | 3.6 |

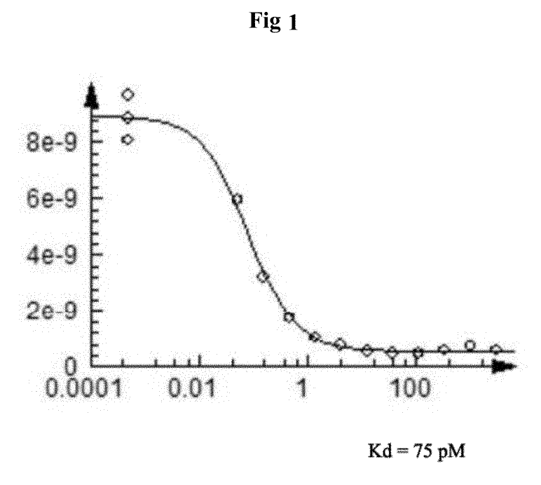

In a separate study, protein kinase profiling of apilimod was conducted to identify kinase targets (DiscoveRx, Fremont, Calif.). A dissociation constant (K_{d}) study was performed using apilimod at increasing concentrations (0.05-3000 nM) against PIKfyve, a known target of apilimod. The experiment was performed in duplicate and the K_{d} was determined to be 0.075 nM (range 0.069-0.081 nM) (FIG. 1).

Next, apilimod was screened against a comprehensive panel of kinases (PIKfyve not included). In total, 456 kinases, including disease-relevant kinases, were assayed for their ability to bind with apilimod. The screening concentration of apilimod was 1 μM, a concentration that is >10,000 times greater than the K_{d} for apilimod against PIKfyve. The results from the screen showed that apilimod did not bind to any of the 456 kinases tested.

Together, these results demonstrate that apilimod binds with high selectivity in cancer cells to a single cellular kinase, PIKfyve.

### Example 2: Apilimod Inhibits Endosome to Lysome Fusion, but not Endocytosis, Via Inhibiting PIKfyve

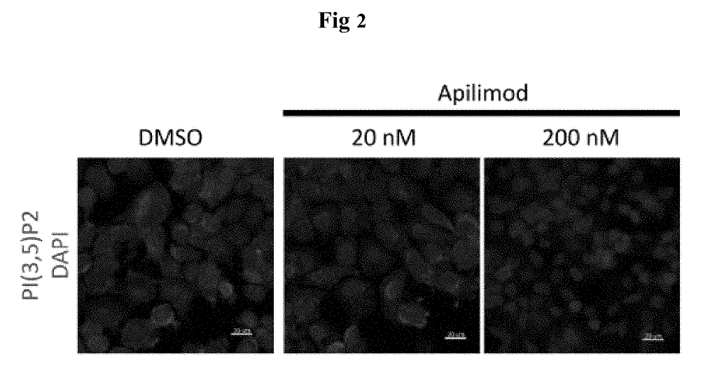

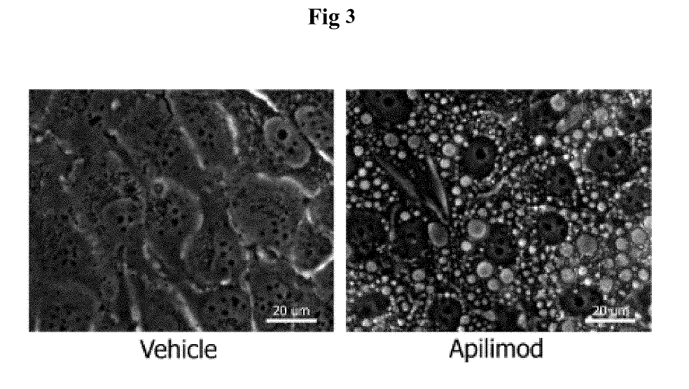

PIKfyve produces the lipid products PI(3,5)P₂ and PI5P, which in turn serve to establish membrane identity and control endolysosomal dynamics. Studies have shown that the PI(3,5)P₂ depletion arising from inhibition of PIKfyve produces a swollen endolysosome phenotype. Based on these studies, we tested the relationship between apilimod, PIKfyve inhibition and endolysosomal dysfunction. As shown in FIG. 2 our experiments demonstrated that apilimod induces approximately a 70% decrease of PI(3,5)P₂ in H4 neuroglioma cells after 24 h and these cells exhibit a striking swollen endolysosomal (vacuole) phenotype (FIG. 3). This phenotype was reversible, and cell lines reverted to normal appearance within 3-4 days after drug removal.

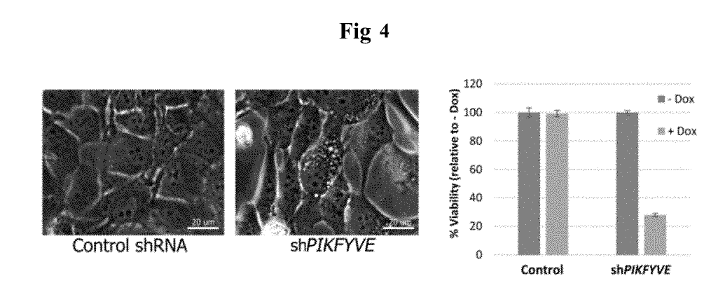

We next tested whether repression of the PIKfyve gene product could recapitulate the cytotoxic effect seen with apilimod in one of the cell lines tested, the H4 neuroglioma cells. As shown in FIG. 4, the induction of a doxycycline-inducible hairpin targeting the PIKFYVE transcript induced a swollen endolysosomal phenotype in these cells.

Together, these results demonstrate that apilimod's inhibition of PIKfyve kinase inhibits endosome to lysome fusion, but not endocytosis.

1. A method for increasing the transcytosis of an agent across a cell, the method comprising contacting the cell with a composition comprising the agent and contacting the cell with an inhibitor of PIKfyve.

2. (canceled)

3. (canceled)

4. The method of claim 1, wherein the inhibitor of PIKfyve is APY0201, YM-201636, apilimod free base, or a pharmaceutically acceptable salt of any of the foregoing.

5. (canceled)

6. The method of claim 1, wherein the inhibitor of PIKfyve is an interfering RNA molecule (RNAi) or an anti-PIKfyve antibody, or antigen binding fragment thereof.

7. (canceled)

8. (canceled)

9. The method of claim 1, wherein the cell is an endothelial cell or an epithelial cell.

10. The method of claim 1, wherein the contacting is ex vivo.

11. The method of claim 1, wherein the agent is a macromolecule and the macromolecule is an antibody, a bioactive peptide, or a therapeutic bacteriophage.

12-15. (canceled)

16. A method for administering a therapeutic agent to the brain of a subject in need thereof, the method comprising administering to the subject (i) a composition comprising the agent and (ii) an inhibitor of PIKfyve.

17. (canceled)

18. The method of claim 16, wherein the composition comprising the agent further comprises a transferrin receptor binding moiety.

19. The method of claim 16, wherein the inhibitor of PIKfyve is APY0201, YM-201636, apilimod free base, or a pharmaceutically acceptable salt thereof.

20. (canceled)

21. The method of claim 16, wherein the inhibitor of PIKfyve is an interfering RNA molecule (RNAi) or an anti-PIKfyve antibody, or antigen binding fragment thereof.

22. The method of claim 18, wherein the transferrin receptor binding moiety comprises an anti-transferrin receptor antibody, or an antigen-binding fragment thereof.

23-26. (canceled)

27. The method of claim 18, wherein the subject in need thereof is a subject in need of treatment for Alzheimer's disease.

28. The method of claim 27, wherein the therapeutic antibody is selected from an anti-β-secretase (BACE1) antibody, an anti-amyloid-β antibody, an anti-tau protein antibody, or an antigen binding fragment of any of the foregoing.

29-31. (canceled)

32. The method of claim 18, wherein the cellular PIKfyve is in the endothelial cells of the blood brain barrier.

33. The method of claim 18, wherein the inhibitor of PIKfyve is administered prior to, at the same time as, or after the composition comprising the macromolecule.

34. (canceled)

35. (canceled)

36. The method of claim 18, further comprising administering to the subject at least one additional active agent.

37. The method of claim 36, wherein the inhibitor of PIKfyve is apilimod and at least one additional active agent is administered in the same composition as the composition comprising apilimod, or in a separate composition from the composition comprising apilimod.

38. The method of claim 37, wherein the at least one additional active agent is a non-therapeutic agent.

39-42. (canceled)

43. The method of claim 38, wherein the non-therapeutic agent is a CYP3A inhibitor.

44. The method of claim 43, wherein the CYP3A inhibitor is ritonavir or cobicistat.
